(19) United States
(12) Patent Application Publication
(10) Pub. No.: US 2007/0225353 A1
(43) Pub. Date: Sep. 27, 2007

(54) Process for Forming Amorphous Atorvastatin

(75) Inventors: Jason Albert Leonard, Concord, NH (US); Peter Robert Rose, Ledyard, CT (US)

Correspondence Address: WARNER-LAMBERT COMPANY, 2800 PLYMOUTH RD, ANN ARBOR, MI, 48105, US

(73) Assignee: Pfizer, Inc.

(21) Appl. No.: 11/578,084

(22) PCT Filed: Apr. 4, 2005
(86) PCT No.: PCT/IB05/00884

§ 371 Date: Oct. 12, 2006

Related U.S. Application Data
(60) Provisional application No. 60/562,948, filed on Apr. 16, 2004.

(51) Int. Cl. A61K 31/40; C07D 207/34

(52) U.S. Cl. 514/423; 548/537

(57) ABSTRACT

Forming amorphous atorvastatin comprises the steps of dissolving atorvastatin in a solvent to form a solution, followed by adding the solution to a mixture comprising a non-solvent and a hydroxylic solvent to afford amorphous atorvastatin.

### FIELD OF THE INVENTION

The invention relates to processes for forming amorphous atorvastatin by precipitating atorvastatin from a solution using a non-solvent containing a hydroxylic solvent.

### BACKGROUND OF THE INVENTION

The conversion of 3-hydroxy-3-methylglutaryl-coenzyme A (HMG-CoA) to mevalonate is an early and rate-limiting step in the cholesterol biosynthetic pathway. This step is catalyzed by the enzyme HMG-CoA reductase. Statins inhibit HMG-CoA reductase from catalyzing this conversion. As such, statins are collectively potent lipid lowering agents.

Atorvastatin calcium is currently sold as Lipitor® having the chemical name [R-(R*,R*)]-2-(4-fluorophenyl)-β,δ-dihydroxy-5-(1-methylethyl)-3-phenyl-4-[(phenylamino)carbonyl]-1H-pyrrole-1-heptanoic acid calcium salt (2:1) trihydrate and the formula

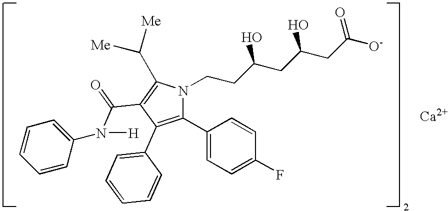

Atorvastatin and pharmaceutically acceptable salts thereof are selective, competitive inhibitors of HMG-CoA reductase. As such, atorvastatin calcium is a potent lipid lowering compound and is thus useful as a hypolipidemic and/or hypocholesterolemic agent, as well as in the treatment of osteoporosis, benign prostatic hyperplasia (BPH) and Alzheimer's disease.

A number of patents have issued disclosing atorvastatin, formulations of atorvastatin, as well as processes and key intermediates for preparing atorvastatin. These include: U.S. Pat. Nos. 4,681,893; 5,273,995; 5,003,080; 5,097,045; 5,103,024; 5,124,482; 5,149,837; 5,155,251; 5,216,174; 5,245,047; 5,248,793; 5,280,126; 5,397,792; 5,342,952; 5,298,627; 5,446,054; 5,470,981; 5,489,690; 5,489,691; 5,510,488; 5,686,104; 5,998,633; 6,087,511; 6,126,971; 6,433,213; and 6,476,235, which are herein incorporated by reference.

Additionally, a number of published International Patent Applications and patents have disclosed crystalline forms of atorvastatin, as well as processes for preparing amorphous atorvastatin. These include: U.S. Pat. No. 5,969,156; U.S. Pat. No. 6,121,461; U.S. Pat. No. 6,605,759; WO 01/36384; WO 02/41834; WO 02/43667; WO 02/43732; WO 02/051804; WO 02/057228; WO 02/057229; WO 02/057274; WO 059087; WO 02/083637; WO 02/083638; WO 03/011826; WO 03/050085; WO 03/07072; and WO 04/022053.

It has been disclosed that the amorphous forms of a number of drugs exhibit different dissolution characteristics and in some cases different bioavailability patterns compared to the crystalline form (Konno T., Chem. Pharm. Bull., 1990;38:2003-2007). For some therapeutic indications one bioavailability pattern may be favored over another.

Variations in dissolution rates can make it advantageous to produce atorvastatin formulations in either crystalline or amorphous forms. For example, for some potential uses of atorvastatin (e.g., acute treatment of patients having strokes as described in Takemoto, M.; Node, K.; Nakagami, H.; Liao, Y.; Grimm, M.; Takemoto, Y.; Kitakaze, M.; Liao, J. K., Journal of Clinical Investigation, 2001; 108(10): 1429-1437) a rapid onset of activity may be highly beneficial in improving the efficacy of atorvastatin.

The preparation of amorphous atorvastatin has been previously disclosed. For example, Lin et al., U.S. Pat. No. 6,087,511 disclose forming amorphous atorvastatin from crystalline atorvastatin. To form amorphous atorvastatin, Lin et al. disclose that crystalline atorvastatin is dissolved in a non-hydroxylic solvent such as tetrahydrofuran. The non-hydroxylic solvent is removed to produce a brittle foam that is broken up by mechanical agitation to afford amorphous atorvastatin.

WO 00/71116 also discloses forming amorphous atorvastatin using a non-hydroxylic solvent.

WO 01/28999 discloses a process for forming amorphous atorvastatin by recrystallization of crude atorvastatin from an organic solvent which comprises dissolving crude amorphous atorvastatin calcium in a lower alkanol containing 2-4 carbon atoms or a mixture of such alkanols under heating. The amorphous atorvastatin calcium is precipitated after cooling.

WO 01/42209 discloses preparing amorphous atorvastatin by precipitating the atorvastatin using a solvent in which atorvastatin is insoluble or very slightly soluble, from a solution of atorvastatin which is provided with a solvent in which atorvastatin is freely soluble. Preferred solvents in which atorvastatin is freely soluble include low molecular weight alcohols, e.g. methanol and ethanol.

The current processes for production of amorphous atorvastatin involve solvents which are not optimal due to toxicity or environmental concerns. In addition, current processes are not optimal in terms of production capabilities and are not suitable for large scale synthesis. Therefore, there remains a continuing need for improved methods for preparation of amorphous atorvastatin.

### SUMMARY OF THE INVENTION

A first aspect of the present invention is a process for forming amorphous atorvastatin comprising the steps of: (a) dissolving atorvastatin in a solvent to form a solution; and b) adding the solution to a mixture comprising a non-solvent and a hydroxylic solvent to afford amorphous atorvastatin.

In a preferred method, the non-solvent that is used to precipitate amorphous atorvastatin calcium comprises an aliphatic or alkane solvent, such as, for example, heptane, heptanes, hexane, and the like in combination with a soluble/miscible hydroxylic solvent, such as, for example, 2-propanol.

We have unexpectedly found that the addition of a small amount of a hydroxylic solvent in the non-solvent can be employed to improve the precipitation and formation of amorphous atorvastatin calcium. More specifically, amorphous material is formed when the atorvastatin calcium is dissolved in a solution containing a freely soluble solvent, and is added to a non-solvent containing small quantities of a hydroxylic solvent. The use of a hydroxylic solvent in the non-solvent mixture provides one or more of the following advantages. The use of a hydroxylic solvent has the additional advantage that the amorphous atorvastatin calcium does not stick to reactor walls which readily occurs when a hydroxylic solvent is not present during the precipitation process. In addition, the small particle size achieved by the precipitation process alleviates the need for a milling step, thereby reducing the number of unit operations in production of the material for commercial use. These process advantages are extremely important in the production of large quantities of amorphous atorvastatin on a commercial or factory scale.

The use of amorphous atorvastatin produced by the process of the present invention in unit dosage forms is disclosed in copending, commonly assigned patent application titled “Pharmaceutical Compositions of Atorvastatin,” (Attorney docket number PC25684, Serial Number ______).

A second aspect of the present invention is a therapeutic package or kit suitable for commercial sale, comprising a container and a therapeutically effective amount of amorphous atorvastatin calcium.

A third aspect of the present invention is a method of using amorphous atorvastatin calcium to treat subjects suffering from hypercholesterolemia and/or hyperlipidemia, osteoporosis, benign prostatic hyperplasia (BPH) and Alzheimer's disease.

The foregoing and other objectives, features and advantages of the invention will be more readily understood upon consideration of the following detailed description of the invention.

### BRIEF DESCRIPTION OF THE DRAWINGS

FIG. 1 shows a powder X-ray diffraction (PXRD) diffractogram of amorphous atorvastatin calcium made in Example 1.

### DETAILED DESCRIPTION OF THE INVENTION

As will be recognized by those skilled in the art, the initial atorvastatin which is dissolved in a suitable solvent may be in any morphological form such as, for example, crystalline or amorphous, as well as disordered crystals, liquid crystals, plastic crystals, mesophases, and the like, or any combination thereof. Atorvastatin may readily be prepared, for example, as described in U.S. Pat. Nos. 4,681,893, 5,273,995 and 5,969,156 which are incorporated herein by reference. The term “atorvastatin” includes the free acid form, salt forms, solvates, hydrates and polymorphs. Pharmaceutically acceptable base addition salts of atorvastatin are formed with metals or amines, such as alkali and alkaline earth metals or organic amines. Examples of metals used as cations are sodium, potassium, magnesium, calcium, and the like. Examples of suitable amines are N,N′-dibenzylethylenediamine, chloroprocaine, choline, diethanolamine, dicyclohexylamine, ethylenediamine, N-methylglucamine, and procaine (see, for example, Berge, S. M., et al., “Pharmaceutical Salts”, J. of Pharm. Sci., 1977; 66:1).

A preferred form of atorvastatin is atorvastatin hemi-calcium salt trihydrate, sold under the tradename LIPITORO®.

The term “alkane” as used herein refers to a straight or branched aliphatic hydrocarbon of from six to ten carbon atoms, such as, for example hexane, heptane, heptanes, and the like.

The term “alkanol” as used herein refers to an “alkane” as defined above of from one to six carbon atoms containing one or more hydroxyl groups, such as, for example, 1-propanol, 2-propanol, and the like.

The term “cycloalkane” as used herein refers to a hydrocarbon ring containing five to eight carbon atoms, such as, for example, cyclohexane, cycloheptane, and the like.

Amorphous atorvastatin calcium is formed by precipitation by a process in which a solution of atorvastatin calcium is added to a non-solvent mixture comprising a non-polar solvent and a hydroxylic co-solvent. Solvents suitable for dissolving atorvastatin calcium include, for example, polar organic solvents in which atorvastatin calcium is soluble. Non-solvents suitable for use in the present process include alkanes and other non-polar and low polarity solvents, such as, for example, hexane, heptane, heptanes, and the like, cycloalkanes, such as, for example, cyclohexane, and the like, as well as other non-polar and low-polar solvents, such as, for example, toluene, isopropyl ether and the like. Hydroxylic solvents suitable for use in the present process include organic solvents containing one or more hydroxyl groups. Solvents suitable for dissolution can be any solvent in which the atorvastatin is soluble. Preferably, atorvastatin has a solubility of at least 1 wt %, and more preferably at least 5 wt % in the dissolving solvent. Preferably, the solvent is also volatile with a boiling point of 150° C. or less. In addition, the solvent should have relatively low toxicity and be able to be removed from the amorphous atorvastatin to a level that is acceptable according to The International Committee on Harmonization (ICH) guidelines. Removal of solvent to this level may require a subsequent processing step such as, for example, tray-drying. Preferred dissolution solvents include, for example, tetrahydrofuran, 2-methyltetrahydrofuran, acetone, methyl ethyl ketone, and the like. Preferred non-solvent mixtures contain a mixture of an alkane or cycloalkane solvent such as, for example, heptane containing a small quantity of a hydroxylic solvent such as, for example, an alkanol having from one to six carbon atoms, for example, 2-propanol.

Thus, in the present process, atorvastatin calcium is dissolved in a solvent in which it is freely soluble. Preferred solvents include, for example, aprotic polar solvents, such as, for example, tetrahydrofuran (THF), 2-methyltetrahydrofuran, acetone, methylethyl ketone, and the like. The most preferred solvent is THF.

The preferred concentrations of atorvastatin in the dissolving solvent are determined by the solubilities of atorvastatin calcium in the dissolving solvent. For THF the range of concentrations suitable for the present process are between about 8 and about 20 ml solvent per gram of atorvastatin calcium. The preferred solvent concentration is about 10 mL of solvent per gram of atorvastatin calcium.

The non-solvent used in the present process is a solvent in which atorvastatin calcium is insoluble or only slightly soluble. Preferred non-solvents include, for example, an alkane, such as, for example, hexane, heptane, heptanes, octane, and the like or a cycloalkane, such as, for example, cyclohexane, in combination with a small quantity of a hydroxylic solvent, such as, for example, an alkanol having from one to six carbon atoms and which is miscible with the non-solvent. The more preferred non-solvent includes hexane, heptane, heptanes, and the like and the more preferred hydroxylic solvent additives include 2-propanol or 1-propanol. The most preferred non-solvent is heptane and the most preferred hydroxylic solvent is 2-propanol. The amount of the hydroxylic solvent present in the non-solvent can range from about 0.5 to about 5% by volume. The preferable amount of hydroxylic solvent is about 1 to about 3 %.

The preferable non-solvent concentration for the precipitation process can range from about 15 mL non-solvent/g of atorvastatin calcium to about 60 mL of non-solvent/g of atorvastatin calcium. The preferred non-solvent concentration is about 30 mL per gram of atorvastatin calcium.

Preferable temperatures for carrying out the precipitation of amorphous atorvastatin calcium is between about 10° C. to about 35° C. The more preferred temperature range is about 15° C. to about 25° C.

The preferable addition mode is to add the solution of atorvastatin calcium to the non-solvent mixture containing an alkane or cycloalkane and a small quantity of the hydroxylic solvent. The strong driving force for precipitation of amorphous atorvastatin calcium is generally provided by adding the solution of atorvastatin calcium to the non-solvent mixture thus generating a higher level of supersaturation. The preferred addition time is to add the solution to the non-solvent over about 15 minutes to about 8 hours. The more preferred addition time is about 1 to about 2 hours.

Preferably, cooling is performed in less than about 1 hour to greater than about 4 hours. The preferred isolation temperature range is between about −10° C. and about 30° C. The most preferred isolation temperature is between about 0° C. to about 20° C. The preferred final stir time prior to isolation is typically less than about 8 hours. The more preferred final stir time is less than about 4 hours.

A key feature of the present invention is the use of a non-solvent containing a small quantity of a hydroxylic solvent that aids in the formation of the precipitated amorphous atorvastatin calcium. Incorporation of the hydroxylic solvent in the non-solvent affords precipitated amorphous atorvastatin calcium particles which do not stick to the reactor walls or other internal components. This is especially important in preparing large scale quantities of amorphous atorvastatin. The addition of a hydroxylic solvent in small quantities provides sufficient hydroxyl groups that prevent the interaction and binding to the reactor internals such as silanol groups in a glass lined reactor.

The amount of crystalline material present in the resulting amorphous product is small. Preferably at least 90 wt %, more preferably at least 95 wt %, and even more preferably at least 99 wt % of the resulting product is amorphous after precipitation by addition of a solution of atorvastatin into the non-solvent mixture. Amorphous material, and the amount of amorphous material present, may be characterized by techniques known in the art such as powder x-ray diffraction crystallography, solid state nuclear magnetic resonance (NMR) spectroscopy, or thermal techniques such as differential scanning calorimetry (DSC).

The present invention relates to the treatment of diseases and conditions in a subject, such as, hyperlipidemia and/or hypercholesterolemia, osteoporosis, benign prostatic hyperplasia (BPH) and Alzheimer's disease with amorphous atorvastatin calcium as described above that may be administered in a solid dosage form containing a pharmaceutically acceptable carrier or diluent and/or contained in a therapeutic package or kit. The kit may include the solid dosage form and a container. Typically, the kit includes directions for administration of the dosage form. The container can be in any conventional shape or form as known in the art, for example, a paper box, a glass or plastic bottle, or a blister pack with individual dosage for pressing out of the back according to a therapeutic schedule.

Other features and embodiments of the invention will become apparent from the following examples which are given for illustration of the invention rather than for limiting its intended scope.

### EXAMPLE 1

Crystalline atorvastatin calcium (U.S. Pat. No. 5,969,156) (1.80 kg) was dissolved in tetrahydrofuran (18 L) by stirring in a jacketed glass reactor with overhead agitation. The THF solution was added over 2 hours to a mixture containing heptanes (55 L) and 2-propanol (1.125 L) in a jacketed glass reactor with overhead agitation at a temperature of between 15° C. and 25° C. The resulting slurry was stirred for 1 hour followed by cooling the slurry to approximately 0° C. to 5° C. over 1 hour. The slurry was stirred at 0° C. to 5° C. for 30 minutes. The precipitated material was isolated on a horizontal plate filter covered with polyethylene cloth by vacuum filtration and dried under vacuum (range 20 to 30 inches vacuum) at about 50° C. to about 60° C. Amorphous atorvastatin calcium (1.6 kg) was collected containing 0.01% THF and 0.8% heptane.

X-Ray Powder Diffraction

The X-ray powder diffraction pattern of amorphous atorvastatin calcium was carried out on a Bruker D5000 diffractometer (Madison, Wis.) equipped with copper radiation (Cu K_{α}). Data were collected from 3.0 to 40.0 degrees in two theta (20) using a step size of 0.04 degrees and a step time of 1.0 seconds. The divergence and scattering slits were set at 1 mm, and the receiving slit was set at 0.6 mm. Diffracted radiation was detected by a Kevex PSI detector. An alumina standard was analyzed to check the instrument alignment. Data were collected and analyzed using Bruker AXS software Version 7.0. Samples were prepared for analysis by placing them in a quartz holder. The sample is typically placed into a quartz holder which has a cavity. It should be noted that Bruker Instruments purchased Siemans; thus, a Bruker D5000 instrument is essentially the same as a Siemans D5000.

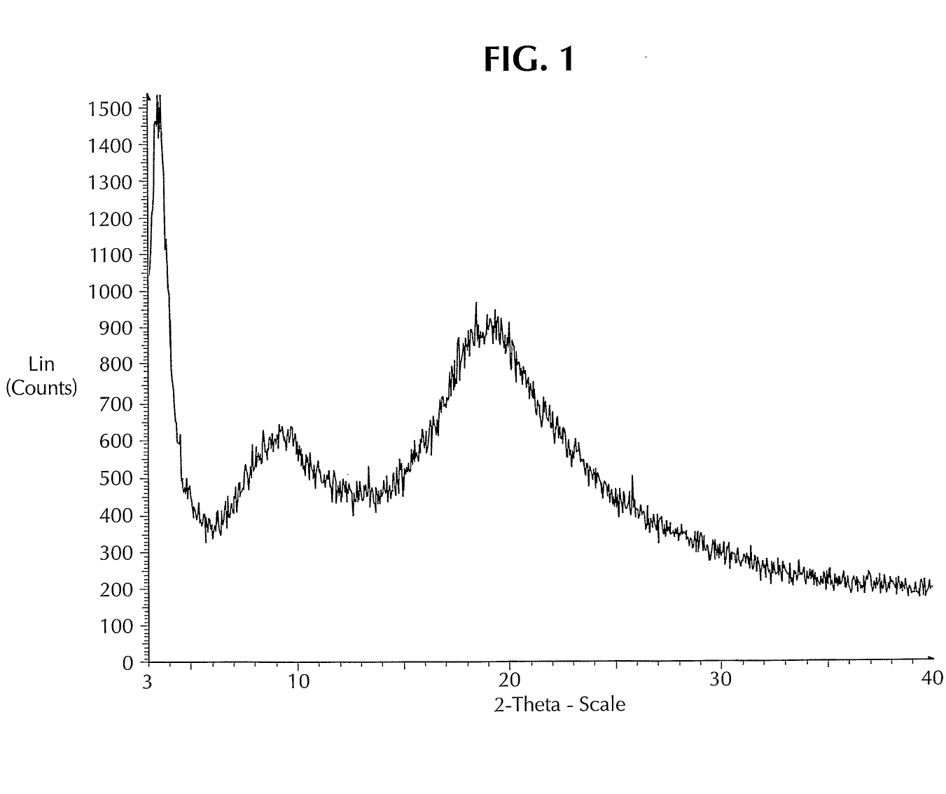

FIG. 1 shows a powder x-ray diffraction of Example 1 showing the material is amorphous.

### EXAMPLE 2

Crystalline atorvastatin calcium (U.S. Pat. No. 5,969,156) (8 g) was dissolved in tetrahydrofuran (80 mL) by stirring in a jacketed glass reactor with overhead agitation. The THF solution was added over 1 hour to a mixture containing heptane (245 mL) and 2-propanol (5 mL) in a jacketed glass reactor with overhead agitation at a temperature of between 15° C. and 25° C. The slurry was cooled to 0° C. to 5° C. The precipitated material was isolated on a ceramic Buchner style funnel covered with a paper filter cloth by vacuum filtration and dried under vacuum (range 20 to 30 inches vacuum) at about 40° C. to about 50° C. Amorphous atorvastatin calcium (7.1 g) was collected.

### EXAMPLE 3

Crystalline atorvastatin calcium (U.S. Pat. No. 5,969,156) (8 g) was dissolved in tetrahydrofuran (80 mL) by stirring in a jacketed glass reactor with overhead agitation. The THF solution was added over 2 hour to a mixture containing heptane (240 mL) and 2-propanol (10 mL) in a jacketed glass reactor with overhead agitation at a temperature of between 15° C. and 25° C. The slurry was cooled to 0° C. to 5° C. over 1.5 hours. The precipitated material was isolated on a ceramic Buchner style funnel covered with a paper filter cloth by vacuum filtration and dried under vacuum (range 20 to 30 inches vacuum) at about 40° C. to about 50° C. Amorphous atorvastatin calcium (6.5 g) was collected.

### EXAMPLE 4

Crystalline atorvastatin calcium (U.S. Pat. No. 5,969,156) (8 g) was dissolved in tetrahydrofuran (80 mL) by stirring in a jacketed glass reactor with overhead agitation. The THF solution was added over 1 hour to a mixture containing heptane (242 mL) and 2-propanol (7.5 mL) in a jacketed glass reactor with overhead agitation at a temperature of between 15° C. and 25° C. The slurry was cooled to 0° C. to 5° C. over about 1 to 2 hours. The precipitated material was isolated on a ceramic Buchner style funnel covered with a paper filter cloth by vacuum filtration and dried under vacuum (range 20 to 30 inches vacuum) at about 40° C. to about 50° C. Amorphous atorvastatin calcium (7.2 g) was collected.

### EXAMPLE 5

Crystalline atorvastatin calcium (U.S. Pat. No. 5,969,156) (8 g) was dissolved in tetrahydrofuran (80 mL) by stirring in a jacketed glass reactor with overhead agitation. The THF solution was added over 1 hour to a mixture containing heptane (248 mL) and 2-propanol (2.5 mL) in a jacketed glass reactor with overhead agitation at a temperature of between 15° C. and 25° C. The slurry was cooled to 0° C. to 5° C. over 1 hour. The precipitated material was isolated on a ceramic Buchner style funnel covered with a paper filter cloth by vacuum filtration and dried under vacuum (range 20 to 30 inches vacuum) at about 40° C. to about 50° C. Amorphous atorvastatin calcium (6.5 g) was collected.

The terms and expressions which have been employed in the foregoing specification are used therein as terms of description and not of limitation, and there is no intention, in the use of such terms and expressions, of excluding equivalents of the features shown and described or portions thereof, it being recognized that the scope of the invention is defined and limited only by the claims which follow.

1. A process for forming amorphous atorvastatin, comprising:
(a) dissolving atorvastatin in a solvent to form a solution ; and
(b) adding the solution to a mixture comprising a non-solvent and a hydroxylic solvent to afford amorphous atorvastatin.

2. The process of claim 1 wherein said solvent in step (a) is a solvent in which atorvastatin is soluble.

3. The process of claim 2 wherein said solvent is selected from the group consisting of tetrahydrofuran, 2-methyltetrahydrofuran, acetone, and methyl ethyl ketone.

4. The process of claim 3 wherein the solvent is tetrahydrofuran.

5. The process of claim 1 wherein said non-solvent in step (b) is selected from the group consisting of an alkane and a cycloalkane.

6. The process of claim 5 wherein said solvent is selected from the group consisting of hexane, heptane, heptanes, octane, and cyclohexane.

7. The process of claim 6 wherein said solvent is heptane.

8. The process of claim 1 wherein said hydroxylic solvent in step (b) is an alkanol containing one to six carbon atoms.

9. The process of claim 8 wherein said solvent is selected from the group consisting of methanol, ethanol, 1-propanol, 2-propanol, 1-butanol, 2-butanol, 2-methyl-2-propanol, 1-pentanol, 2-pentanol, 1-hexanol, 2-hexanol, and 3-hexanol.

10. The process of claim 9 wherein said solvent is 2-propanol.

11. The process of claim 8 wherein about 0.5 to about 5% by volume of said hydroxylic solvent is present in the non-solvent.

12. The process of claim 11 wherein about 1 to about 3% by volume of said hydroxylic solvent is present in the non-solvent.

13. The process of claim 1 wherein said non-solvent concentration is about 15 ml to about 60 ml of non-solvent to one gram of atorvastatin calcium.

14. The process of claim 13 wherein said non-solvent concentration is about 30 ml to one gram of atorvastatin calcium.

15. The process of claim 1 wherein in step (b) precipitation of amorphous atorvastatin is carried out at about 10° C. to about 35° C.

16. The process of claim 15 wherein in step (b) precipitation of amorphous atorvastatin is carried out at about 15° C. to about 25° C.

17. The process of claim 1 wherein the solution of atorvastatin is added to the mixture comprising a non-solvent and a hydroxylic solvent over about 15 minutes to about 8 hours.

18. The process of claim 17 wherein the addition is over about 1 to about 2 hours.

19. The process of claim 1 wherein the mixture is cooled to about −10° C. to about 30° C. for about less than one hour to greater than about four hours.

20. The process of claim 19 wherein the mixture is cooled at about 0° C. to about 20° C. for about four hours.

21. The process of claim 1 which affords at least 90 weight percent amorphous atorvastatin.

22. The process of claim 21 which affords at least 95 weight percent amorphous atorvastatin.

23. The process of claim 22 which affords at least 99 weight percent amorphous atorvastatin.
